(19) United States
(12) Patent Application Publication
(10) Pub. No.: US 2025/0078967 A1
(43) Pub. Date: Mar. 6, 2025

(54) PATIENT INFORMATION PROVISION METHOD, PATIENT INFORMATION PROVISION APPARATUS, AND COMPUTER-READABLE RECORDING MEDIUM

(71) Applicant: D&LIFE, Seoul (KR)

(72) Inventors: Yu Sub SUNG, Seoul (KR); Tae Won KIM, Seongnam-si, Gyeonggi-do (KR); Shin Kyo YOON, Seoul (KR)

(21) Appl. No.: 18/722,391

(22) PCT Filed: Dec. 2, 2022
(86) PCT No.: PCT/KR2022/019526

§ 371 Date: Jun. 20, 2024

(30) Foreign Application Priority Data
Dec. 21, 2021 (KR) 10-2021-0184190

(51) Int. Cl. G16H 10/60; G16H 30/20

(52) U.S. Cl. CPC G16H 10/60; G16H 30/20

(57) ABSTRACT

There is provided a method for providing patient information. The method comprises receiving image data of a medical record of a patient; converting the image data of the medical record into text data; extracting item data from the text data; evaluating reliability of the extracted item data; and providing patient information regarding the extracted item data if the evaluated reliability is equal to or greater than a pre-determined threshold value. According to the method for providing patient information, there is provided a have the effect of effectively extracting necessary information from unstandardized medical records.

### TECHNICAL FIELD

Embodiments relate to a patient information provision method and apparatus for providing information on a current condition of a patient.

This research was supported by Korea Health Industry Development Institute (KHIDI), funded by the Korea government (Ministry of Health and Welfare (MOHW)) (No. 1465033623, Development of a Communication-based Medical Device Platform for Precision Cancer Care: SMART Cancer Care-Bot)

### BACKGROUND ART

Generally, copies of medical records are issued upon patient request in hospitals. Patient's disease information and conditions are recorded in medical records. Patients can ascertain information on diseases and their conditions through medical records issued by hospitals.

However, since medical records are written in the technical terminology prescribed by clinicians, there is a problem in that it is difficult for patients to easily interpret the technical terminology.

In addition, since medical records are not written in a standardized format depending on the type, there is a problem in that it is difficult for patients to ascertain their diseases and conditions.

### DETAILED DESCRIPTION OF INVENTION

### Technical Problems

In order to solve the above-mentioned problems, an object of embodiments is to provide a method and apparatus for providing patient information to effectively deliver information on patient's conditions through medical records.

### Technical Solution

In accordance with an aspect of the present disclosure, there is provided a method for providing patient information, the method comprises: receiving image data of a medical record of a patient; converting the image data of the medical record into text data; extracting item data from the text data; evaluating reliability of the extracted item data; and providing patient information regarding the extracted item data if the evaluated reliability is equal to or greater than a pre-determined threshold value.

The evaluating reliability of the item data may include comparing the extracted item data with preset standard item data; and evaluating the reliability of the item data by determining whether essential items of the preset standard item data are included in the extracted item data.

The method may further comprise re-extracting item data using a pre-trained machine learning model if the essential items of the standard item data are not included in the extracted item data.

The comparing the extracted item data with standard item data may be performed using at least one of a rule-based model, a Burt model, a decision tree model, and a neural network model.

The medical record may include at least one of a pathology report, a genetic examination report, a regional imaging examination report (CT and MRI), a nuclear medicine imaging examination report (PET/CT), and a prescription.

The item data may include at least one of the name, date of birth, age, sex, treatment history, biopsy results, genetic examination results, imaging examination results, prescription drug, and medicines of the patient.

The patient information may include at least one of patient's diagnosis, stage, whether surgery is performed, surgery name, customized treatment information, treatment process, and information on clinical trials in which the patient is able to participate.

Additionally, in accordance with another aspect of the present disclosure, there is provided a device for providing patient information, the device comprises: a memory configured to store one or more instructions; and a processor configured to execute the one or more instructions stored in the memory, wherein the instructions, when executed by the processor, cause the processor to: receive image data of a medical record of a patient; convert the image data of the medical record into text data; extract item data from the text data; evaluate reliability of the extracted item data; and provide patient information regarding the extracted item data if the evaluated reliability is equal to or greater than a pre-determined threshold value.

The processor may be configured to compare the extracted item data with preset standard item data, and evaluate the reliability of the item data by determining whether essential items of the preset standard item data are included in the extracted item data.

The processor may be configured to re-extract item data using a pre-trained machine learning model if the essential items of the standard item data are not included in the extracted item data.

The processor may be configured to compare the extracted item data with standard item data using at least one of a rule-based model, a Burt model, a decision tree model, and a neural network model.

The medical record may include at least one of a pathology report, a genetic examination report, a regional imaging examination report (CT and MRI), a nuclear medicine imaging examination report (PET/CT), and a prescription.

The item data may include at least one of the name, date of birth, age, sex, treatment history, biopsy results, genetic examination results, imaging examination results, prescription drug, and medicines of the patient.

The patient information may include at least one of patient's diagnosis, stage, whether surgery is performed, surgery name, customized treatment information, treatment process, and information on clinical trials in which the patient is able to participate.

Additionally, in accordance with another aspect of the present disclosure, there is provided a non-transitory computer readable storage medium storing computer executable instructions, wherein the instructions, when executed by a processor, cause the processor to perform a method for providing patient information, the method comprise: receiving image data of a medical record of a patient; converting the image data of the medical record into text data; extracting item data from the text data; evaluating reliability of the extracted item data; and providing patient information regarding the extracted item data if the evaluated reliability is equal to or greater than a pre-determined threshold value.

Additionally, in accordance with another aspect of the present disclosure, there is provided computer program including computer executable instructions stored in a non-transitory computer readable storage medium, wherein the instructions, when executed by a processor, cause the processor to perform a method for providing patient information, the method comprise: receiving image data of a medical record of a patient; converting the image data of the medical record into text data; extracting item data from the text data; evaluating reliability of the extracted item data; and providing patient information regarding the extracted item data if the evaluated reliability is equal to or greater than a pre-determined threshold value.

### Advantageous Effects

Embodiments have the effect of effectively extracting necessary information from unstandardized medical records.

Additionally, embodiments have the effect of effectively ascertaining a patient's current condition using information extracted from medical records.

In addition, embodiments have the effect of effectively informing a patient of his or her condition by providing information extracted from medical records to a user.

Additionally, embodiments have the effect of improving the accuracy of information extracted from medical records by evaluating the reliability of the information extracted from the medical records.

### DESCRIPTION OF DRAWINGS

FIG. 1 is a block diagram showing a patient information provision system according to an embodiment.

FIG. 2 is a diagram showing types of medical records provided by a server included in the patient information provision system according to an embodiment.

FIG. 3 is a block diagram showing the patient information provision apparatus included in the patient information provision system according to an embodiment.

FIG. 4 is a flowchart showing a method of providing patient information performed by the patient information provision apparatus of FIG. 3

FIG. 5 and FIG. 6 are flowcharts showing detailed steps of the method of providing patient information shown in FIG. 4.

### BEST MODE FOR CARRYING OUT THE INVENTION

The advantages and features of the present invention, and the methods of achieving them, will become apparent by referring to the embodiments described in detail below in conjunction with the accompanying drawings. However, the present invention is not limited to the embodiments disclosed herein and can be implemented in various forms. These embodiments are provided to make the disclosure of the present invention thorough and to fully convey the scope of the invention to those skilled in the art, and the scope of the present invention is defined only by the claims.

In describing the embodiments of the present invention, specific descriptions of well-known functions or configurations will be omitted for clarity and conciseness where they are not essential to understanding the embodiments. The terms used herein are defined in consideration of the functions in the embodiments of the present invention and may vary depending on the user, operator's intention, or custom. Therefore, the definitions should be based on the entire content of this specification.

The present invention may be subject to various modifications and may include several embodiments, specific embodiments being illustrated in the drawings and described in detail. However, this is not intended to limit the present invention to specific embodiments, and it should be understood to include all modifications, equivalents, and substitutes included within the spirit and scope of the present invention as defined by the appended claims.

Terms including ordinals such as first, second, etc., may be used to describe various components, but these components are not limited by such terms. These terms are used only to distinguish one component from another.

When a component is described as being “connected” or “coupled” to another component, it may be directly connected or coupled to the other component or intervening components may be present.

Hereinafter, embodiments will be described in detail with reference to the accompanying drawings.

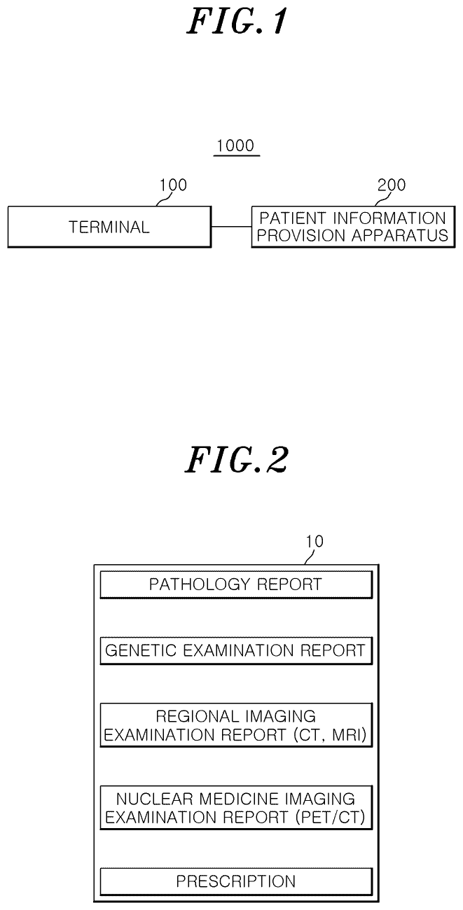

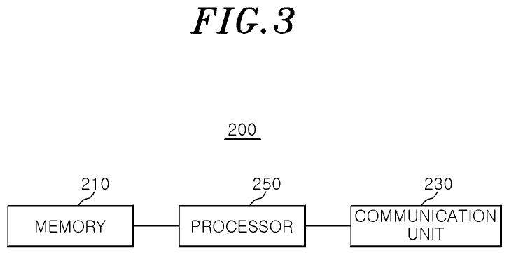

FIG. 1 is a block diagram showing a patient information provision system according to an embodiment, and FIG. 2 is a diagram showing types of medical records provided by a server included in the patient information provision system according to an embodiment.

Referring to FIG. 1, the patient information provision system according to an embodiment may include a terminal 100 and a patient information provision apparatus 200.

The terminal 100 may be a terminal used by a patient or a carer. Image data regarding medical records may be stored in the terminal 100.

Image data regarding medical records may be an image obtained by a patient or a carer scanning a record sheet issued by a hospital or capturing the record sheet using a camera. Alternatively, image data regarding medical records may be received from a medical institution, such as a hospital. As shown in FIG. 2, a medical record may include, but is limited to, pathology reports, genetic examination reports, regional imaging examination reports (CT and MRI), nuclear medicine imaging examination reports (PET/CT), prescriptions, etc.

A pathology report is data obtained by examining samples of cancer tissue characteristics, such as tissues, cells, and organs, and may include, but is not limited to, information such as a sample type, a tissue collection site, and a subtype. A regional imaging examination report may include, but is not limited to, CT and MRI data and a report on results of imaging examination for each cancer region. A nuclear medicine imaging examination report may include, but is not limited to, information such as PET/CT data, examination methods, and reading results. Prescription information may include information on prescribed medicine, such as prescription date, a prescription issuance date, a prescribed medicine name, single dose, the number of daily administrations, the total number of days of administration, total dosage, and an administration method. A genetic examination report may include, but is not limited to, information such as examination methods, examination items, and genetic examination results.

The patient information provision apparatus 200 may receive image data regarding a medical record from the terminal 100. Alternatively, the patient information provision apparatus 200 may receive image data regarding a medical record from a server provided in a hospital or a medical institution in addition to the terminal.

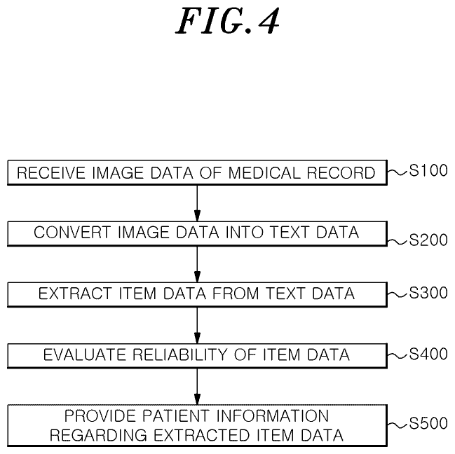

FIG. 3 is a block diagram showing the patient information provision apparatus included in the patient information provision system according to an embodiment.

As shown in FIG. 3, the patient information provision apparatus 200 may include a memory 210, a communication unit 230, and a processor 250.

The memory 210 may store various types of data for the overall operation of the patient information provision apparatus 200, such as a control program for processing of the processor 250 or data transmission. Specifically, the memory 210 may store a plurality of application programs executed in the patient information provision apparatus 200, and data and instructions for the operation of the patient information provision apparatus 200.

The memory 210 may include, but is not limited to, magnetic storage media or flash storage media.

The communication unit 230 is connected to the terminal 100 and may provide a communication interface capable of communicating with the terminal 100 using a plurality of communication methods.

The communication unit 230 may be a device that includes hardware and software necessary to transmit and receive signals such as control signals or data signals through wired or wireless connections with other network devices.

The communication unit 230 may perform communication using not only 3G, LTE, and 5G, but also a low power wireless network (LPWN) and a low power wide area network (LPWAN) such as NB-IoT, LoRa, SigFox, and LTE-CAT1.

The communication unit 230 may perform communication through a communication method using a wireless local area network (LAN) such as WiFi 80211a/b/g/n as well as a wired LAN. In addition, the communication unit 230 may communicate with the terminal 100 using communication methods such as NFC and Bluetooth.

The processor 250 is a type of central processing device and can control the patient information provision apparatus 200.

The processor 250 may include any type of device capable of processing data. Here, “processor” may mean, for example, a data processing device built into hardware that has a physically structured circuit in order to perform a function represented by code or instructions included in a program. Examples of data processing devices built into hardware include a microprocessor, a central processing unit (CPU), a processor core, a multiprocessor, an application-specific integrated circuit (ASIC), and a field programmable gate array (FPGA), but are not limited thereto.

Hereinafter, a method of providing patient information performed by the processor 250 of the patient information provision apparatus 200 will be described.

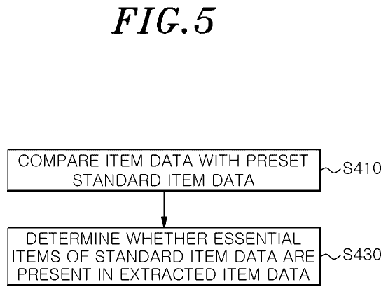

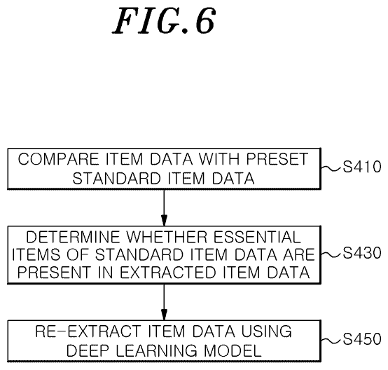

FIG. 4 is a flowchart showing a method of providing patient information performed by the patient information provision apparatus of FIG. 3, and FIG. 5 and FIG. 6 are flowcharts showing detailed steps of the method of providing patient information shown in FIG. 4.

As shown in FIG. 4, when image data of a medical record is received from the terminal, the processor 250 of the patient information provision apparatus 200 may recognize the image data (S100).

Medical records may include, but are not limited to, pathology reports, genetic examination reports, regional imaging examination reports (CT, MRI), nuclear medicine imaging examination reports (PET/CT), prescriptions, etc.

A pathology report is data obtained by examining samples of cancer tissue characteristics, such as tissues, cells, and organs, and may include, but is not limited to, information such as a sample type, a tissue collection site, and a subtype. A regional imaging examination report may include, but is not limited to, CT and MRI data and a report on results of imaging examination for each cancer region. A nuclear medicine imaging examination report may include, but is not limited to, information such as PET/CT data, examination methods, and reading results. Prescription information may include information on prescribed medicine, such as prescription date, a prescription issuance date, a prescribed medicine name, single dose, the number of daily administrations, the total number of days of administration, total dosage, and an administration method. A genetic examination report may include, but is not limited to, information such as examination methods, examination items, and genetic examination results.

The image data regarding the medical record may be data scanned or captured using a terminal of a patient.

The processor 250 may convert the recognized image data into text data (S200). The processor 250 may convert the image data into text data using optical character recognition (OCR) technology.

The processor 250 may extract item data from the text data (S300). The item data is data for analyzing a patient's condition and may include, but is not limited to, at least one of the patient's name, diagnosis, prescription, and medicine.

The processor 250 may evaluate the reliability of the extracted item data (S400). If the processor 250 determines that the extracted item data is reliable data, the processor 250 may provide patient information on the extracted item.

More specifically, as shown in FIG. 5, the processor 250 may compare the item data with preset standard item data stored in the memory (S410). The processor 250 may compare the item data with the standard item data using any one of a rule-based model, a Burt model, a decision tree model, and a neural network model.

The processor 250 may determine whether essential items of the standard item data are present in the extracted item data (S430).

The processor 250 may determine whether essential items exceeding a preset threshold value are present in the extracted item data. The preset threshold value may be set such that similarity between essential items of the standard item data and the extracted item data is 90% to 100%. The threshold value may be preset by a user.

If the processor 250 determines that the essential items of the standard item data are present in the extracted item data, the processor 250 may provide patient information on the extracted item (S500). The patient information may include description data regarding the item data. Here, the description data may be data for interpreting the values of item data written in the medical record and explaining the interpreted meaning to the patient. The description data may be data such as text, image, video, or voice.

The patient information may include at least one of a patient's diagnosis, stage, whether surgery has been performed, a surgery name, customized treatment information, treatment process, and information on clinical trials in which the patient can participate based on interpretation of the item data values recorded in the medical record, but is not limited thereto.

The processor 250 may provide the patient information to the terminal. Alternatively, the processor 250 may provide the patient information to a server installed in a hospital or to a terminal of a medical worker, and the types are not limited.

On the other hand, as shown in FIG. 6, the processor 250 may compare the item data with preset standard item data stored in the memory (S410). The processor 250 may compare the item data with the standard item data using any one of a rule-based model, a Burt model, a decision tree model, and a neural network model.

The processor 250 may determine whether essential items of the standard item data are present in the extracted item data (S430).

The processor 250 may determine whether essential items exceeding a preset threshold value are present in the extracted item data. The preset threshold value may be set such that similarity between the essential items of the standard item data and the extracted item data is 90% to 100%. The threshold value may be preset by the user.

If the processor 250 determines that the essential items of the standard item data are not present in the extracted item data, the processor 250 may re-extract item data (S450). Here, if the similarity between the essential items of the standard item data and the extracted item data is, for example, less than 90%, the processor 250 may determine that the essential items of the standard item data are not present in the extracted item data.

The processor 250 may re-extract item data using an artificial intelligence model previously stored in the memory. The artificial intelligence model may be a pre-trained model using training item data.

When the item data is re-extracted, the processor 250 may reevaluate the reliability of the re-extracted item data by re-performing steps S410 and S430.

If the processor 250 determines that essential items of the standard item data are present in the re-extracted item data, the processor 250 may provide patient information on the re-extracted item (S500). The patient information may include description data regarding the item data. Here, the description data may be data for interpreting the values of item data written in the medical record and explaining the interpreted meaning to the patient. The description data may be data such as text, image, video, or voice.

The patient information may include at least one of the patient's diagnosis, stage, whether surgery has been performed, surgery name, customized treatment information, treatment process, and information on clinical trials in which the patient can participate based on interpretation of the item data values recorded in the medical record.

The processor 250 may provide the patient information to the terminal 100. Alternatively, the processor 250 may provide the patient information to a server installed in a hospital or to a terminal of a medical worker, and the types are not limited.

The patient information provision apparatus according to the embodiment has the effect of improving the accuracy of information extracted from medical records.

Various embodiments of this document can be implemented as software (e.g., a program) containing instructions stored on machine-readable storage media (e.g., memory, either internal or external). The machine, such as a computer, can retrieve the stored instructions from the storage media and operate according to the retrieved instructions. The machine can include an electronic device according to the disclosed embodiments. When the instructions are executed by the control unit, the control unit can perform the functions corresponding to the instructions either directly or by using other components under its control. The instructions can include code generated or executed by a compiler or an interpreter. The machine-readable storage media can be provided in the form of non-transitory storage media. Here, non-transitory means that the storage media does not include signals and is tangible, but does not distinguish whether the data is stored permanently or temporarily on the storage media.

According to an embodiment, the methods according to the various embodiments disclosed in this document can be provided in a computer program product.

In one embodiment, a computer-readable recording medium storing a computer program includes instructions for a processor to perform operations comprising: receiving image data of a patient's medical record, converting the image data of the medical record into text data, extracting item data from the text data, evaluating the reliability of the extracted item data, and providing patient information for the extracted item data if the evaluated reliability exceeds a threshold.

In another embodiment, a computer program stored on a computer-readable recording medium includes instructions for a processor to perform operations comprising: receiving image data of a patient's medical record, converting the image data of the medical record into text data, extracting item data from the text data, evaluating the reliability of the extracted item data, and providing patient information for the extracted item data if the evaluated reliability exceeds a threshold.

Although the embodiments have been described with reference to the drawings and examples, it will be understood by those skilled in the art that various modifications and changes can be made within the technical idea of the embodiments described in the following claims.

1. A method for providing patient information, the method comprising:
receiving image data of a medical record of a patient;
converting the image data of the medical record into text data;
extracting item data from the text data;
evaluating reliability of the extracted item data; and
providing patient information regarding the extracted item data if the evaluated reliability is equal to or greater than a pre-determined threshold value.

2. The method of claim 1, wherein the evaluating reliability of the item data includes:
comparing the extracted item data with preset standard item data; and
evaluating the reliability of the item data by determining whether essential items of the preset standard item data are included in the extracted item data.

3. The method of claim 2, further comprises re-extracting item data using a pre-trained machine learning model if the essential items of the standard item data are not included in the extracted item data.

4. The method of claim 2, wherein the comparing the extracted item data with standard item data is performed using at least one of a rule-based model, a Burt model, a decision tree model, and a neural network model.

5. The method of claim 1, wherein the medical record includes at least one of a pathology report, a genetic examination report, a regional imaging examination report (CT and MRI), a nuclear medicine imaging examination report (PET/CT), and a prescription.

6. The method of claim 1, wherein the item data includes at least one of the name, date of birth, age, sex, treatment history, biopsy results, genetic examination results, imaging examination results, prescription drug, and medicines of the patient.

7. The method of claim 1, wherein the patient information includes at least one of patient's diagnosis, stage, whether surgery is performed, surgery name, customized treatment information, treatment process, and information on clinical trials in which the patient is able to participate.

8. A device for providing patient information, the device comprising:
a memory configured to store one or more instructions; and
a processor configured to execute the one or more instructions stored in the memory, wherein the instructions, when executed by the processor, cause the processor to:
receive image data of a medical record of a patient;
convert the image data of the medical record into text data;
extract item data from the text data;
evaluate reliability of the extracted item data; and
provide patient information regarding the extracted item data if the evaluated reliability is equal to or greater than a pre-determined threshold value.

9. The device of claim 8, wherein the processor is configured to compare the extracted item data with preset standard item data, and evaluate the reliability of the item data by determining whether essential items of the preset standard item data are included in the extracted item data.

10. The device of claim 9, the processor is configured to re-extract item data using a pre-trained machine learning model if the essential items of the standard item data are not included in the extracted item data.

11. The device of claim 9, the processor is configured to compare the extracted item data with standard item data using at least one of a rule-based model, a Burt model, a decision tree model, and a neural network model.

12. The device of claim 8, wherein the medical record includes at least one of a pathology report, a genetic examination report, a regional imaging examination report (CT and MRI), a nuclear medicine imaging examination report (PET/CT), and a prescription.

13. The device of claim 1, wherein the item data includes at least one of the name, date of birth, age, sex, treatment history, biopsy results, genetic examination results, imaging examination results, prescription drug, and medicines of the patient.

14. The device of claim 1, wherein the patient information includes at least one of patient's diagnosis, stage, whether surgery is performed, surgery name, customized treatment information, treatment process, and information on clinical trials in which the patient is able to participate.

15. A non-transitory computer readable storage medium storing computer executable instructions, wherein the instructions, when executed by a processor, cause the processor to perform a method for providing patient information, the method comprising:
receiving image data of a medical record of a patient;
converting the image data of the medical record into text data;
extracting item data from the text data;
evaluating reliability of the extracted item data; and
providing patient information regarding the extracted item data if the evaluated reliability is equal to or greater than a pre-determined threshold value.

16. The non-transitory computer readable storage medium of claim 15, wherein the evaluating reliability of the item data includes:
comparing the extracted item data with preset standard item data; and
evaluating the reliability of the item data by determining whether essential items of the preset standard item data are included in the extracted item data.

17. The non-transitory computer readable storage medium of claim 16, further comprises re-extracting item data using a pre-trained machine learning model if the essential items of the standard item data are not included in the extracted item data.

18. The non-transitory computer readable storage medium of claim 2, wherein the comparing the extracted item data with standard item data is performed using at least one of a rule-based model, a Burt model, a decision tree model, and a neural network model.

19. The method of claim 1, wherein the medical record includes at least one of a pathology report, a genetic examination report, a regional imaging examination report (CT and MRI), a nuclear medicine imaging examination report (PET/CT), and a prescription.

20. The method of claim 1, wherein the item data includes at least one of the name, date of birth, age, sex, treatment history, biopsy results, genetic examination results, imaging examination results, prescription drug, and medicines of the patient.
